(19) United States
(12) Patent Application Publication
(10) Pub. No.: US 2002/0114742 A1
(43) Pub. Date: Aug. 22, 2002

(54) Test chamber

(75) Inventors: Satoshi Takahashi, Hitachinaka (JP); Taisaku Seino, Tsuchiura (JP); Kenji Yasuda, Tokyo (JP)

Correspondence Address: Crowell & Moring LLP, The Evenson, McKeown, Edwards & Lenahan, Intellectual Property Law Gr., 1001 Pennsylvania Avenue, N.W., Washington, DC, 20004-2595, US

(73) Assignee: Hitachi, Ltd.

(21) Appl. No.: 10/077,902

(22) Filed: Feb. 20, 2002

(30) Foreign Application Priority Data
Feb. 20, 2001 (JP) 2001-043263

(51) Int. Cl. G01N 31/22; G01N 33/52; B01L 3/00

(52) U.S. Cl. 422/102; 422/58

(57) ABSTRACT

Disclosed is a test chamber capable of performing a stain step without moving the test chamber itself under a microscope. The test chamber for observing and testing a smeared specimen, with cells and a tissue specimen comprises an observation portion (fixing portion) for observing the specimen and reagent storage portions, each storing a different reagent (stain liquid, rinse liquid) for stain from others therein, and sends the reagent to the specimen in response to stimulation from the outside to perform the stain.

### BACKGROUND OF THE INVENTION

1. Field of the Invention

The present invention relates to a test chamber for performing cyto-pathological diagnosis and for observing and examining specimens of pathology tissues and the like by use of a microscope, particularly to a simple test chamber having a stain function in the chamber itself.

2. Descriptions of the Related Arts

Examinations such as pathological and cyto-pathological diagnoses are important examination methods in performing diagnosis of cancer and the like. The examinations and the diagnoses are usually performed after tissues are fixed on glass slides or cells and the like are evently spread on glass slides (preparation of smears), and specimens are prepared, followed by treatments such as stains. Various kinds of techniques have been known as the stains, and Papanicolaou stain, Hematoxylin-Eosin stain, Giemsa stain and the like have been applied in accordance with objects. For example, in the Papanicolaou stain, treatment steps including nuclear stain by Gill-Hematoxylin, rinsing by water and ethanol, decolorizing by alcohol (containing hydrochloric acid), rinsing, coloring of nuclei by alcohol (containing ammonia), rinsing, cytoplasmic stain (OG-6), rinsing, cytoplasmic stain (EA), rinsing, dehydration and clearing are performed, whereby cells are stained and used for cyto-pathological diagnosis.

Automation apparatuses for staining cells fixed on slide glasses in such a manner have been already on the market, and are described in Japanese Examined Patent Publication No. Hei 6 (1994)-27682 and the like. Examples of the slide glass that is effective for preparing specimens before the stain are described in Japanese Examined Patent Publication No. Hei 7 (1995)-69253, Japanese Patent Laid-Open No. Hei 9 (1997)-61723 and the like.

Besides the foregoing cyto-pathological diagnosis, a method has been recently developed, in which, by use of DNA probes hybridized to virus genes, cancer-related genes and the like, the target genes existing in tissues or cells are directly detected. The target genes in the sample are hybridized and caught by use of the fluorophore-labeled DNA probe, and the target genes are detected by fluorescent measurements.

In the cyto-pathological diagnosis, existence of abnormality and the like are decided depending on shapes of the cells, sizes of nuclei and cytoplasms, and relations between the cells. The existence of the target gene DNA can be detected using the DNA probe quantitatively. The existence of the genes shows important information that there are risk factors of disease. In the case of virus infection, the infection can be decided before symptoms become apparent, and more reliable cures will be possible. However, the existence of the genes and appearance of the disease do not always accord with each other, generally, and usual diagnoses and observation of shapes of cells and the like are necessary.

Accordingly, more effective diagnoses will be possible by combining the detection results of the genes using the DNA probe and the results of the cyto-pathological diagnosis. Note that the detection of the genes using the DNA probe and the sequence measurement in the cyto-pathological diagnosis are not generally performed. The detection of the genes in the cells is usually performed by use of fluorescent detection after hybridizing the fluorophore-labeled DNA probe. The cyto-pathological diagnosis is performed by measurements of light transmission images after the general stains as described above. Since hybridization and the general stain are performed according to different procedures, these are not usually performed simultaneously but performed separately. For this reason, the specimen after hybridization is first subjected to the fluorescent detection, and then the specimen is stained, followed by the light transmission image measurement, so that an operation in which the specimen is detached from the apparatus and the specimen is set up in a stain apparatus is necessary, and hence troublesome. Although there is a method in which stain is performed for another specimen prepared from the same sample and the light transmission image measurement is performed, this method has a problem that the images of both specimens cannot be made to correspond to each other and a comparison of the images is difficult.

### SUMMARY OF THE INVENTION

An object of the present invention is to solve the foregoing problems and to provide a test chamber capable of simply staining a specimen for cyto-pathological diagnosis.

Another object of the present invention is to provide a test chamber capable of simply staining a specimen after hybridization.

The above-described objects can be achieved by allowing the test chamber itself fixing the specimen thereto to possess a specimen treatment function such as a stain function.

Specifically, a first aspect of the test chamber of the present invention comprises: an observation portion for observing a fixed specimen by use of transmission light; a plurality of reagent storage portions for storing reagent used for a treatment of the specimen fixed to the observation portion, at least a part of walls of each of the reagent storage portions being made of an elastic material; and passages for allowing the reagent storage portions and the observation portion to communicate with each other.

This test chamber can be used for observing and examining the smears (specimen spread cells), the tissue specimen and the like. As the reagent used for the treatment of the specimen, stain liquid for staining the specimen and rinse liquid are included therein. This test chamber sends the selected reagent to the specimen in the observation portion by pressing and thrusting the wall portion of the reagent storage portion, which is constituted of an elastic material (for example, rubber-like material), and can perform stain of the specimen in the test chamber. When pressing of the wall portion of the reagent storage portion, which is constituted of the elastic material, is released, the reagent after being used returns to the reagent storage portion and is stored therein.

The observation portion can be constituted so as to communicate with the open air via the passage having a capacity for accepting an excess of the reagent that is sent from the reagent storage portion to the observation portion and flows out therefrom.

A second aspect of the test chamber of the present invention comprises: an observation portion for observing a fixed specimen by use of transmission light; a plurality of reagent storage portions for storing reagent used for a treatment of the specimen fixed to the observation portion, at least a part of each of the reagent storage portions being sealed by a stickable material; a liquid absorber for absorbing the reagent; and passages for allowing the reagent storage portions and the observation portion to communicate with each other; and a passage for allowing the observation portion and the liquid absorber to communicate with each other.

The observation portion of the test chamber of the present invention is sealed by a transparent member, whereby a space filled with the reagent is formed in the observation portion. Sealing of the observation portion can be performed by a transparent sealing member onto which adhesive is coated.

The test chamber of the present invention can be provided with a transparent plate for fixing the specimen, which is freely detachable from the observation portion.

The transparent plate for fixing the specimen can be made of a slide glass or a material similar to the slide glass. By using the detachable transparent plate for fixing the specimen, a method can be adopted, in which the transparent plate for fixing the specimen is attached/detached to/from the test chamber, cells and the like are smeared and fixed onto the transparent plate outside the test chamber, and a slice of tissue is fixed thereto, followed by an re-attachment of the transparent plate to the test chamber. Thus, handling of the specimen can be facilitated. Moreover, the transparent plate for fixing the specimen can serve also as a transparent plate sealing the observation portion.

The reagent storage portion can hold the reagent in a liquid absorption material. By allowing the liquid absorption material to hold the reagent, it is possible to prevent a drip of a solution and the like, and handling of the reagent can be facilitated.

The test chamber of the present invention should be preferably provided with discrimination means for discriminating reagent sets stored in the plurality of reagent storage portions.

The discrimination means for discriminating the reagent sets can be constituted as a marker made up of barcodes, colors and the like, or formed to a shape of the test chamber itself.

According to the present invention, the stain step can be simply performed without moving the test chamber under the microscope, and the treatment of the waste liquid is facilitated. Moreover, the specimen after the hybridization can be stained simply, and the comparison of the gene localization and the cell image can be performed easily.

### BRIEF DESCRIPTION OF THE DRAWINGS

FIG. 1 is a schematic section view showing an example of a test chamber according to the present invention.

FIGS. 2(a) and 2(b) are schematic views showing an example of a body of the test chamber of the present invention.

FIG. 3 is a schematic section view of the test chamber assembled in examining a specimen.

FIG. 4 is a schematic view showing a constitution example of a measurement apparatus using the test chamber of the present invention.

FIG. 5 is a schematic view showing a constitution example of a liquid sending unit.

FIG. 6 is a schematic view showing another constitution example of the liquid sending unit.

FIG. 7 is a schematic section view showing another example of the body of the test chamber of the present invention.

FIGS. 8(a) and 8(b) are schematic views showing still another example of the body of the test chamber of the present invention.

FIG. 9 is a schematic section view of a test chamber assembled in examining the specimen.

FIG. 10 is a perspective view obtained by partially disassembling the test chamber of the present invention.

FIG. 11(b) is a section view of the test chamber taken along the line C-C′ of FIG. 10, and FIG. 11(a) is a section view of the test chamber taken along the line D-D′ of FIG. 11(b).

FIGS. 12(a) and 12(b) are explanatory views showing another example of the body used for the test chamber of this embodiment.

FIG. 13 is a section view showing a structure of the test chamber assembled at the time of measuring.

FIGS. 14(a) and 14(b) are explanatory views showing another example of the body used for the test chamber of this embodiment.

FIG. 15 is a section view showing a structure of the test chamber assembled at the time of measuring.

### DETAILED DESCRIPTION OF THE PREFERRED EMBODIMENTS

Hereinafter, embodiments of the present invention will be described with reference to the accompanying drawings.

[Embodiment 1]

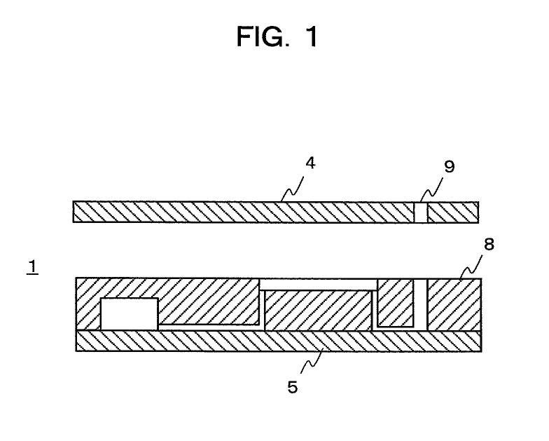

FIG. 1 is a schematic section view showing an example of a test chamber according to the present invention. This test chamber 1 is comprises: a body 8; a lower sealing member 5 adhered to a lower plane of the body 8; and an upper sealing member 4 adhered to an upper plane of the body 8. The upper sealing member 4 is fixedly adhered to the upper plane of the body 8 after a sample such as cells and tissues is fixed to a fixing portion of the test chamber.

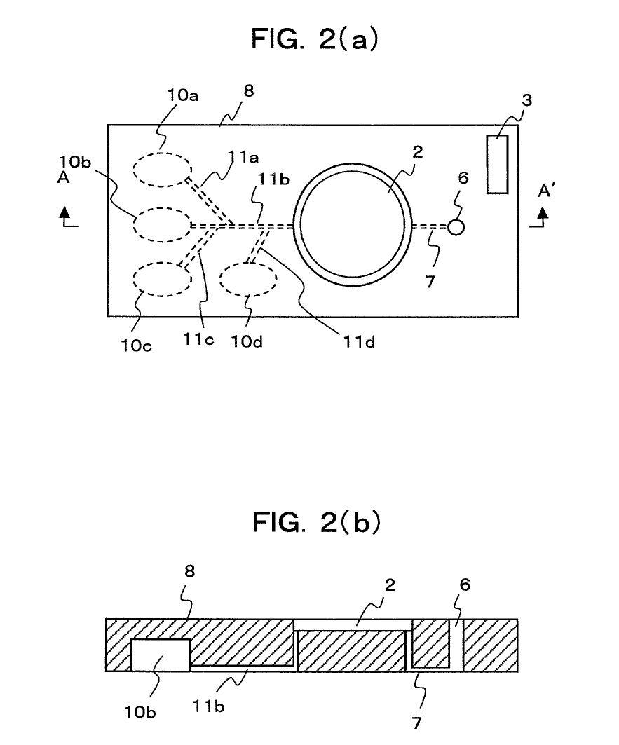

FIGS. 2(a) and 2(b) are schematic views showing an example of the body 8. FIG. 2(a) is a plan view of the body 8, and FIG. 2(b) is a section view taken along the line A-A′ of FIG. 2(a). The body 8 of the test chamber has a size approximately equal to slide glass. The body 8 of the test chamber comprises a fixing portion 2 placed in its center, the fixing portion smearing a sample thereon. The fixing portion 2 shows a concave shape relative to the surface of the body 8, and the sample is smeared on the fixing portion 2, and fixed thereonto. Moreover, the fixing portion 2 serves also as an observation portion. Concave reagent storage portions 10a to 10d for holding various reagents are provided on the rear side of the body 8. The reagent storage portions 10a to 10d and the fixing portion 2 are made to communicate with each other by passages (grooves) 11a to 11d, respectively.

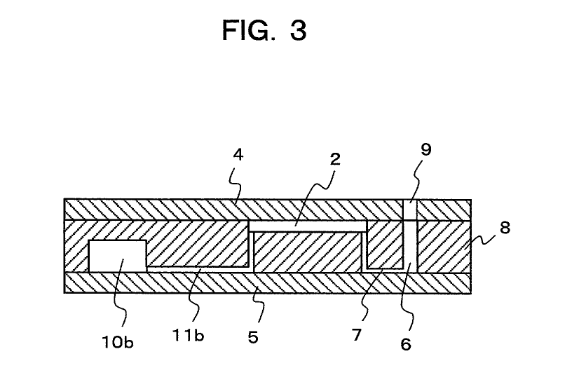

FIG. 3 is a schematic section view showing a state where the test chamber is assembled in examining a specimen. In the reagent storage portions 10a to 10d, necessary reagent such as stain liquid and rinse liquid soaked into a sponge and the like is previously housed, and the reagent storage portions 10a to 10d are sealed by the lower sealing member 5 so that liquid leakage does not occur. A transparent material offering elasticity is used for the lower sealing member 5. Alternatively, the lower sealing member 5 may be formed of an elastic material only in the neighborhood of each of the reagent storage portions 10a to 10d, and portions of the lower sealing member 5 except the neighborhood of each reagent storage portion 10 may be formed of transparent glass adhered thereto. Thus, by pressing the sealing member of each of the reagent storage portions 10a to 10d, the stain liquid soaked into the sponge passes through each of the corresponding passages 11a to 11d to reach the fixing portion 2, where stain and rinse can be performed. Note that, in the drawing, the four reagent storage portions are shown for convenience of the illustration. However, the reagent storage portions are provided by the number required for the stain.

The sample is smeared onto the fixing portion 2 to be fixed thereto. Thereafter, the fixing portion 2 is sealed with the upper sealing member 4. By sealing the fixing portion 2 with the upper sealing member 4, it is possible to prevent the reagent that has reached the fixing portion 2 from leaking to the outside of the test chamber 1. The upper sealing member 4 is formed of a transparent material such as cover glass, and adhered to the test chamber 1 in areas except than the fixing portion 2 by adhesive. Note that the space in the fixing portion 2 communicates with the open air through the passage 7 and the pore 6, which are provided in the body 8, and the pore 9 of the upper sealing member 4 provided at a position coincident with the pore 6, and enables the reagent to move. The passage 7 and the pore 6 provided in the body 8 have a function as a buffer passage. Specifically, when the sample is treated by use of the reagent stored in the reagent storage portion, the reagent is stored in the pore 6 of the body 8 even if the reagent fills the space of the fixing portion 2 and overflows the space, and thus leakage of the reagent to the outside of the test chamber 1 is prevented. By pushing the sealing portion of each of the reagent storage portions 10a to 10d, the reagent fills the space of the fixing portion 2, and the reagent is absorbed in each of the reagent storage portions 10a to 10d when pushing of the sealing member of each of the reagent storage portions 10a to 10d is stopped.

The marker 3, for example, a barcode, for discriminating the reagent sets held in the reagent storage portions 10a to 10d is pasted to the upper plane of the body 8 of the test chamber 1. The marker 3 is not limited to the barcode, and may be identified by color-coding the reagent sets. The discrimination of the reagent sets may be performed by changing the shape of the test chamber itself without using the marker 3.

In the case of an observation of only a stain image, the upper plane of the body 8 having the fixing portion 2 to which the sample is fixed is sealed by the upper sealing member 4, and then the body 8 is set onto a microscope, thus observing the sample. A computer-controlled piston should be provided at a position corresponding to each of the reagent storage portions 10a to 10d of the test chamber 1. The piston is allowed to move upward and downward in the preset order, and a specific reagent storage portion of the test chamber 1 is pressed and the reagent is extruded. Thus, the reagent is guided to the fixing portion 2. When the piston is allowed to return to the original position, the lower sealing portion 5 that has been pushed by the piston to be deformed restores its shape to the original one, a negative pressure is generated in the reagent storage portion, and the reagent in the fixing portion 2 returns to the reagent storage portion. The above described steps are repeated necessary times, thus staining the sample. Thereafter, the light transmission image of the sample fixed to the fixing portion 2 is measured by the microscope, and the measurement results are used for diagnosis.

In another embodiment of the present invention, the sample is smeared on the fixing portion 2 and fixed thereto by an ordinary method. Subsequently, fluorophore-labeled DNA probes are hybridized with the sample fixed to the fixing portion 2 by the ordinary method. In this state, an opening of the fixing portion 2 of the body 8 is sealed by the upper sealing member 4. In this case, a fluorescence observation is performed for the sample, and a fluorescent image of the sample is fetched, thus detecting existence of the target genes. Sorts of the hybridized DNA probe are not limited to one, but plural kinds of DNA probes can be used. At this time, since the labeled fluorophores are plural in number, the fluorescent image is measured with a plurality of wavelengths. Next, after the measurement of the fluorescent image, the reagent is sent to the fixing portion 2 of the test chamber 1 from each of the reagent storage portions 10a to 10d thereof in the predetermined order in a similar manner to the above, and the sample is stained. The stain image is measured by the transmission light. The fluorescent image of the DNA probe and the stain image thereof are displayed so as to superpose one upon another, or displayed so as to be placed side by side. These images are provided to a checking expert (eg. Cyto technologists, Pathologists and other) as information for diagnosis.

Although setting of the test chamber onto the microscope may be performed in the same manner as the case of the ordinary glass slide, the test chamber may be set vertically on its edge, and the fluorescent image and the light transmission image may be acquired from the lateral direction. In this case, if the reagent storage portion is disposed at a position vertically below the fixing portion 2, sending of the reagent is facilitated, and hence the rinsing step and the like are facilitated.

When a slice of tissue besides the smear is fixed to the fixing portion 2, the same steps as the above can be carried out.

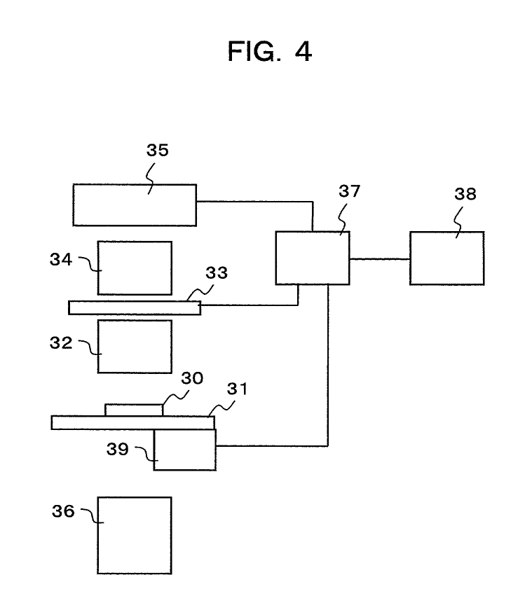

FIG. 4 is a schematic view showing a constitution example of the measurement apparatus using the test chamber 1 of this embodiment. With respect to the test chamber 30 placed to the sample stage 31, the reagent storage portions of the test chamber 30 are sequentially pressed by the liquid sending unit 39 attached to the sample stage 31, and the reagent included in each reagent storage portion is sent, and then the stain operation is executed. Thereafter, the sample is illuminated by the illumination unit 36, and the transmission light transmitted through the sample is processed by collection lens 32, the color filter changer 33 and the focusing lens 34, and the transmission sample image is formed. The transmission sample image is detected by the CCD camera 35. These steps are controlled by the control/data processing unit 37. In this embodiment, in order to obtain a colored transmission sample image by use of the monochrome cooled CCD camera, the color filter changer 33 is used. R, G and B single-color filters are switched from one to another by the color filter changer 33, and the respective monochrome images are measured. The colored image is obtained by synthesizing the obtained monochrome images according to an ordinary method. The synthesized image is displayed on the monitor 38. Note that a color CCD camera may be used in stead of the color filter changer 33 and the monochrome CCD camera.

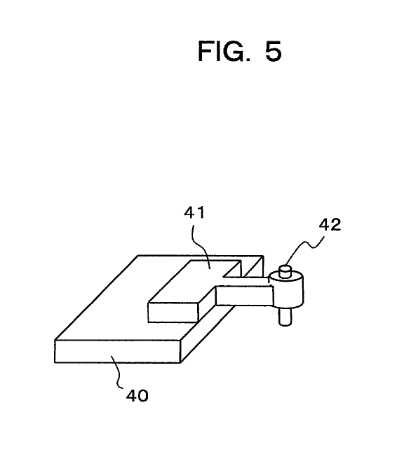

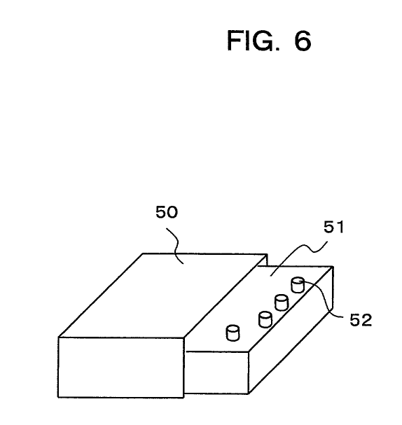

FIGS. 5 and 6 are schematic views showing constitution examples of the liquid sending unit 39. In the liquid sending unit shown in FIG. 5, the X and Y-moving portion 41 is held on the base 40, and the piston unit 42 is disposed at its tip end. The X and Y-moving portion 41 is moved by the control/data processing unit 37 shown in FIG. 4 so that the tip end of the piston unit 42 is moved to the position of the reagent storage portion of the test chamber 1. Thereafter, the piston unit 42 is driven, thus pressing the reagent storage portion.

The liquid sending unit shown in FIG. 6 has a structure in which the plurality of piston units 52 are disposed on the moving stage 51 storaged in the housing portion 50. In the measurement, the moving stage 51 is moved according to demand, and the plurality of piston units 52 disposed on the moving stage 51 are made to be coincident with the respective reagent storage portions of the test chamber. Thus, the pressing operation to press the reagent storage portions can be performed. Note that the plurality of piston units 52 are previously disposed so that the respective piston units 52 are coincident with the respective positions of the reagent storage portions.

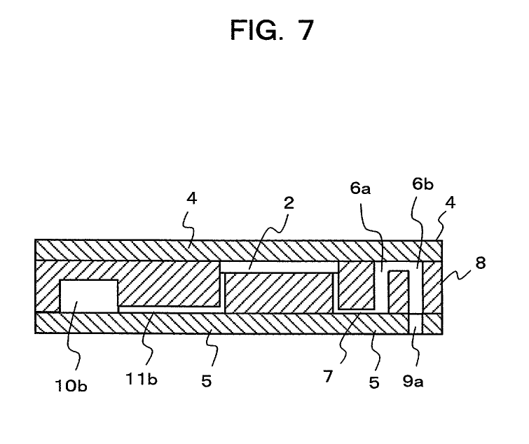

In the examples shown in the drawings, the test chamber 1 is designed so that the space of the fixing portion 2 thereof communicates with the open air through the pore 9 provided in the upper sealing member 4. The pore for allowing the space of the fixing portion 2 to communicate with the open air can be also provided in the lower sealing member 5. FIG. 7 shows a constitution example of the body in the case where the pore 9a for allowing the space of the fixing portion 2 to communicate with the open air is provided in the lower sealing member 5. FIG. 7 corresponds to FIG. 3. As shown in FIG. 7, the two pores 6a and 6b penetrating through the body 8 are provided therein, and the space of the fixing portion 2 is allowed to communicate with the pore 6a by the passage 7, and the pores 6a and 6b are connected to each other by a groove provided on the upper sealing member 4. Then, in the lower sealing member 5, the pore 9a is provided at a position coincident with the pore 6b of the body 8. With this structure, it is also possible to allow the space of the fixing portion 2 to communicate with the open air through the pore 9a provided in the lower sealing member 5. Further, the lower sealing member 5 may provide the opening. The opening, which is almost the same size as the fixing portion 2, is positioned right under the fixing portion 2, and is avoided the passages 7, 11a to 11d. In this case, the lower sealing member 5 can be made of materials which is not transparent, because the illumination lights pass through the opening region.

[Embodiment 2]

In the embodiment 1, the test chamber 1 and the fixing portion 2 are united with each other. However, it is not always necessary to unite the test chamber 1 and the fixing portion 2. A glass plate may be used as the fixing portion, which has a size in accordance with the concave shape of the observation portion of the body. In this case, since the fixing portion is freely detachable from the test chamber, it is possible to perform the operations including the smear, the fixation and the hybridization of the sample in another place except the test chamber.

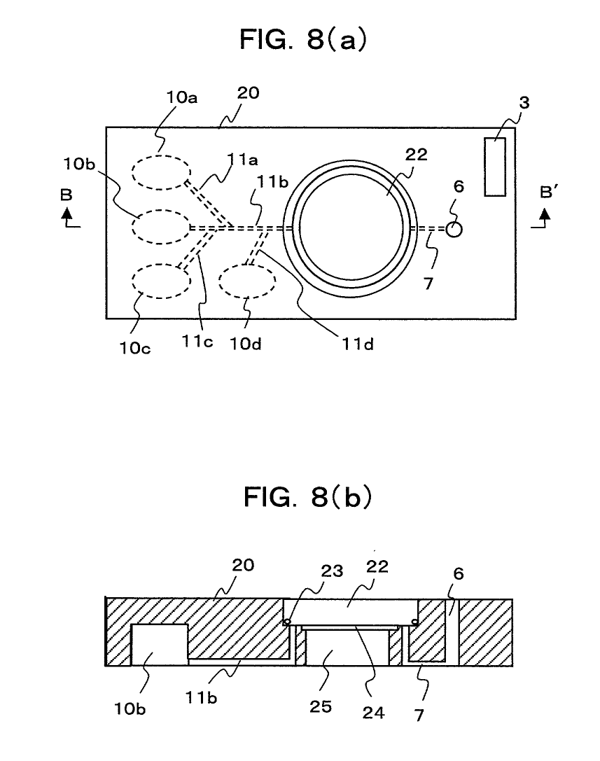

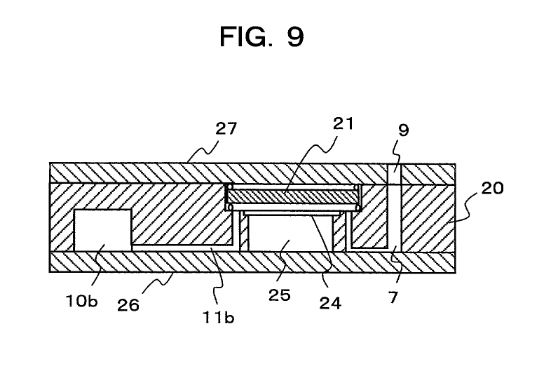

FIGS. 8(a) and 8(b) are schematic views showing another example of the body used for the test chamber of this embodiment. FIG. 8(a) is a plan view, and FIG. 8(b) is a section view taken along the line B-B′ of FIG. 8(a). FIG. 9 is a section view showing a structure of the test chamber which is assembled in the measurement. For facilitating understanding of the structure, in FIGS. 8(a) and 8(b) and FIG. 9, the same constituent components having the equivalent functions to those of FIGS. 2(a) and 2(b) and FIG. 3 are denoted by the same reference numerals, and description of the embodiment 2 will be made.

The test chamber comprises the body 20, the lower sealing member 26 adhered to the lower plane of the body 20 and the upper sealing member 27. When the test chamber is shipped, the lower sealing member 26 is adhered to the lower plane of the body 20, and the upper sealing member 27 is separated from the body 20. When the measurement is performed, a user using this test chamber attaches the fixing portion 21 in the test chamber, and thereafter adheres the upper sealing member 27 to the upper plane of the body 20.

The fixing portion 21 is freely detachable from the test chamber. A glass plate is used as the fixing portion 21, which has a size in accordance with the shape of the concave portion 22 of the body 20, and cells and the like are smeared and fixed thereto. Fluorophore-labeled DNA probes are hybridized with the sample, if necessary. The body 20 of the test chamber has the concave portion 22 storaging glass plate of the fixing portion 21 onto which the sample is smeared and fixed. The rubber-made O-ring 23 for pressing the glass plate of the fixing portion 21 is positioned at the lower portion of the concave portion 22. The glass plate 24 for an observation and the cavity 25 are positioned below the O-ring 23.

Similarly to the embodiment 1, the concave reagent storage portions 10a to 10d for storing various reagents are provided on the rear of the body 20. The reagent storage portions 10a to 10d and the concave portion 22 communicate with each other by the passages (grooves) 11a to 11d. Necessary reagent such as stain liquid and rinse liquid soaked into sponge is stored in the reagent storage portions 10a to 10d, and the reagent storage portions 11a to 10d are sealed by the lower sealing member 26. The portion of the lower sealing member 26 corresponding to the cavity 25 is opened. A sheet offering a rubber-like property is used as the lower sealing member 26, and the sheet is adhered to the lower plane of the body 20. Thus, similarly to the embodiment 1, by pushing the sealing portion of each of the reagent storage portions 10a to 10d, the stain liquid soaked into the sponge in the reagent storage portion passes through each of the corresponding passages 11a to 11d to reach the concave portion 22 where the fixing portion 21 is positioned. Thus, the stain and the rinse of the sample is made possible. The reagent storage portions may be disposed by the number required.

In the measurement, the glass plate of the fixing portion 21 onto which the sample is smeared is inserted in the concave portion 22 of the body 20, and the upper sealing member 27 is sealed from above. A transparent adhesive tape and the like can be used as the upper sealing member 27. Sealing can be made also by use of a glass plate as the upper sealing member 27. In this case, a structure can be adopted, in which an O-ring and the like are disposed between the glass plate of the upper sealing member 27 and the glass plate of the fixing portion 21 so as to prevent leakage of the liquid. Further, the upper seal member 27 may comprise the opening corresponding to the cavity 25 and the glass plate 24. In this case, the upper seal member 27 can be made of materials which is not transparent.

The same operations as those of the embodiment 1 are made possible by the test chamber of this embodiment. Moreover, according to the test chamber of this embodiment, since the operations including the smear, the fixation and the hybridization of the sample such as cells can be performed at a position except the test chamber, these operations can be performed easily.

According to the test chamber shown in the embodiment 1 or 2, since the stain liquid and the like are not thrown out to the outside of the test chamber, the test chamber is handled easily, and disposal of waste liquid becomes unnecessary. Furthermore, since the reagents are used separately for each test chamber, it is possible to perform a stable measurement without influences of contamination of the plurality of samples, the contamination being caused in an ordinary automation staining apparatus when the samples are immersed in a stain liquid tank. Still furthermore, the operations by use of the measurement apparatus such as a microscope are possible, and the stain step can be simply performed without moving the test chamber after the fluorescent measurement. For this reason, a relation between the fluorescent image and the light transmission image becomes clear, and the superpositive display of the fluorescent image and the light transmission image is easily performed. Localizations of the fluorophore-labeled DNA probes can be compared with the cell images, and more precise analysis will be made possible.

[Embodiment 3]

Another example of the test chamber according to the present invention will be described. In the embodiments 1 and 2, the examples in which the reagent soaked into the sponge is stored in the reagent storage portions were described. While this method is effective because of its capability of preventing a drip of a solution, the reagent itself can be also stored in the reagent storage portion.

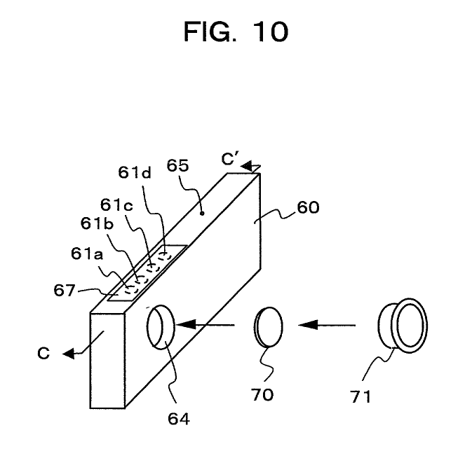

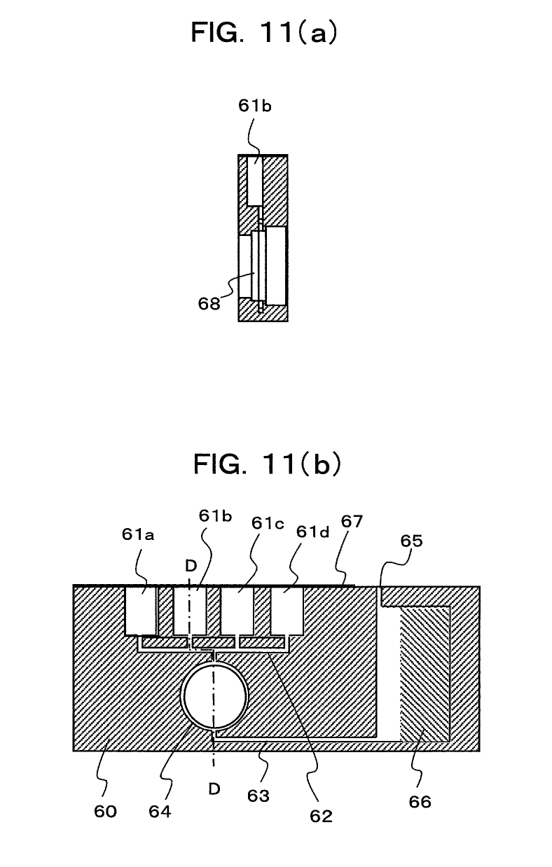

FIG. 10 is a perspective view obtained by partially disassembling the test chamber 60 of this embodiment. FIG. 11(b) is a section view of the test chamber taken along the line C-C′ of FIG. 10. FIG. 11(a) is a section view of the test chamber taken along the line D-D′ of FIG. 11(b).

As shown in FIGS. 11(a) and 11(b), the reagent storage portions 61a, 61b, 61c and 61d are disposed at an upper portion of the test chamber 60, and reagent in each reagent storage portion flows into the test portion 64 via the passage 62. The reagent then passes through the passage 63 and then reaches the liquid absorber 66 to be absorbed there. In the test portion 64, the glass plate 68 is provided, and, as shown in FIG. 10, cells and the like are smeared onto the glass plate (fixing member) 70 separately prepared, followed by necessary treatments similarly to the embodiment 2. The glass plate 70 is inserted in the test portion 64, and then fixed thereto by the fixing member 71. Thus, the test chamber 60 is assembled so as to create a passage between the glass plate 68 and the fixing member 70. The upper portion of each of the reagent storage portions is sealed by the sealing member 67 so that the liquid in the reagent storage portion does not drop. Sending of the reagents in the reagent storage portions 61a to 61d is performed by dropping the reagent naturally from holes punched with a needle at necessary positions of the sealing member 67. Note that, in order to send the reagent more certainly, pressurization should be performed with the air from the holes opened. To perform the pressurization, an apparatus may be adopted, in which a needle-like seal breaker and an air exhaustion portion in stead of the piston unit of the apparatus described in FIGS. 5 and 6 are provided.

As described above, in the test chamber 60 of this embodiment, the reagents are supplied to the reagent storage portions 61a to 61d, which are then sealed up so that the air does not enter thereinto. In this apparatus in this embodiment, the test chamber is set vertically on its edge, and the fluorescent image and the light transmission image are acquired from the lateral direction. The reagent storage portions 61a to 61d are disposed so as to be positioned vertically above the test portion 64 for the sample, and when the reagent is guided into the fixing portion 70 disposed at the test portion 64, the hole is opened in the sealing portion of the necessary reagent storage portion by the needle or the like, and then the reagent is allowed to drop therefrom naturally. The liquid as the reagent reaches the fixing portion 70 of the test portion 64 via the passage 62. The test portion 64 communicates with the liquid absorber 66 through the passage 63. The liquid absorber 66 has a structure in which water absorbing polymer or the like is stuffed therein to prevent leakage of waste liquid to the outside by absorbing the waste liquid. The liquid absorber 66 has the air hole 65 in its upper portion so that the reagents stored in the respective reagent storage portions 61a to 61d flows smoothly.

According to the test chamber of this embodiment, since the stain liquid and the like are not thrown out to the outside of the test chamber similarly to the embodiments 1 and 2, handling of the test chamber becomes facilitated, and a treatment for the waste liquid becomes unnecessary. Moreover, since the reagents are used separately for each test chamber, it is possible to perform the stable measurement without influences of contamination of the plurality of samples, the contamination being caused in an ordinary automation staining apparatus when the samples are immersed in a stain liquid tank. Still furthermore, the operations by use of a microscope are possible, a relation between the fluorescent image and the light transmission image is clear, and the superpositive display of the fluorescent image and the light transmission image is easily performed. Localizations of the fluorophore-labeled DNA probes can be compared with the cell images, and more precise analysis will be made possible

[Embodiment 4]

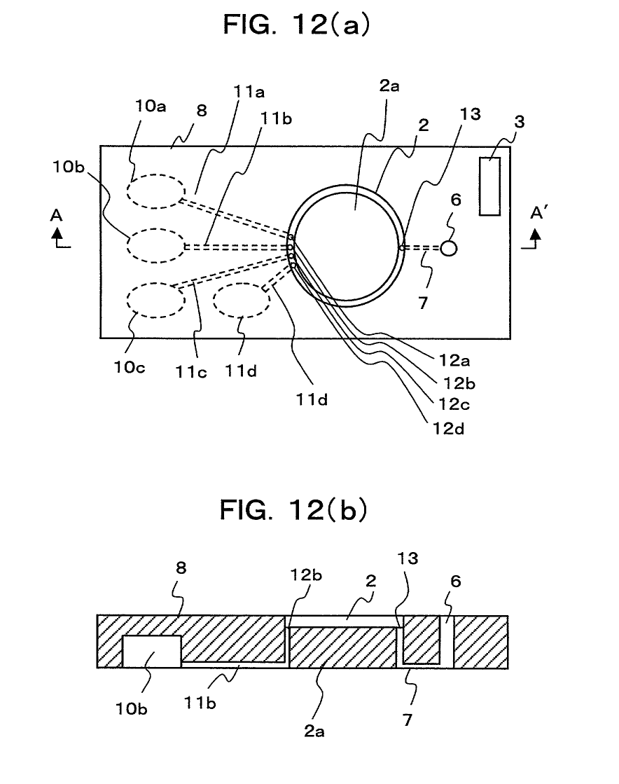

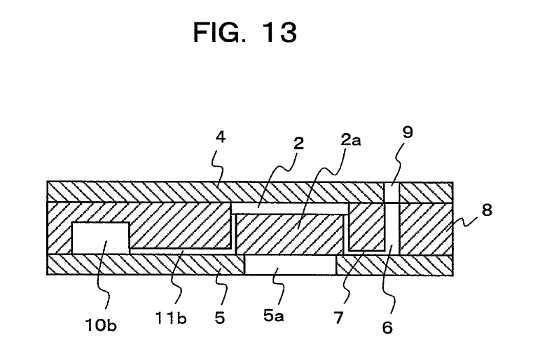

FIGS. 12(a) and 12(b) are explanatory views showing another example of the body used for the test chamber of this embodiment. FIG. 12(a) is a top view, and FIG. 12(b) is a section view taken along the line A-A′ of FIG. 12(a). Moreover, FIG. 13 is a section view showing a structure of the test chamber assembled at the time of measuring. To facilitate understanding of this embodiment, the same function portions of FIGS. 12(a), 12(b) and 13 as those in FIGS. 2 and 3 are denoted by the same reference numerals in FIGS. 2 and 3 and described by use of the same reference numerals.

A material of the fixing portion 2 of the body 8 may be different from those of portions other than the fixing portion 2. A fixing portion stage 2a having a portion of the fixing portion 2 to be smeared is made of a transparent glass circular plate, and a portion of the body 8 other than the fixing portion 2 is prepared by plastic or a metal. Both of them can be also adhered to each other. In this case, working of a concave portion is easy.

Furthermore, with respect to the passages 11a to 11d of the reagent, ports of them can be also provided in the fixing portion, respectively. Reagent introduction/suction ports 12a to 12d are the ones of the fixing portion respectively corresponding to the passages 11a to 11d. The reagent is introduced from the reagent introduction/suction ports 12a to 12d to the fixing portion and sucked thereinto. A reagent drain port 13 is the one on the fixing portion communicated with a hole 6. Surplus reagent and the like are exhausted from the reagent drain port 13.

An opening portion 5a is provided in a lower sealing member 5. The opening portion 5a is disposed approximately just below the fixing portion stage 2a in assembling the test chamber. Thus, influences of a lower sealing member 5 can be removed in the optical measurement. An opaque material can be used for the lower sealing member 5.

[Embodiment 5]

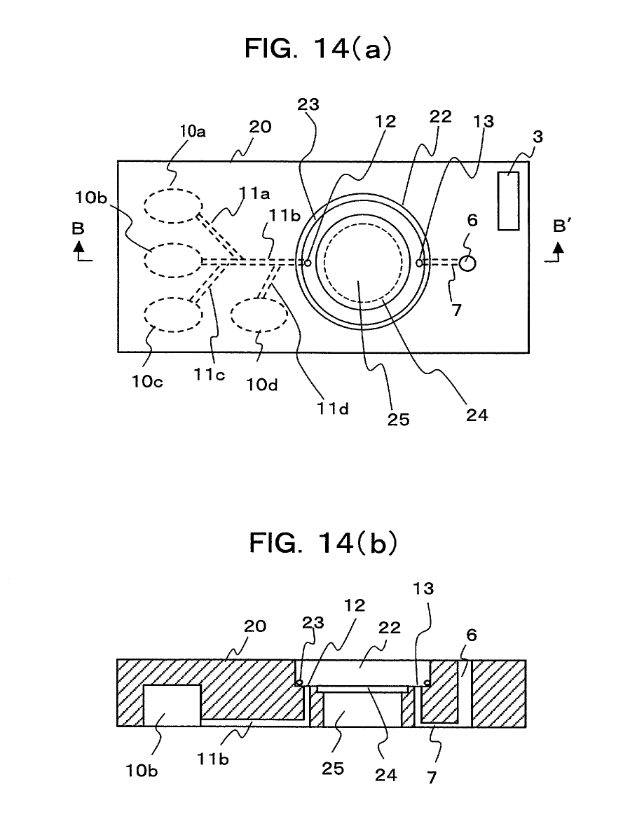

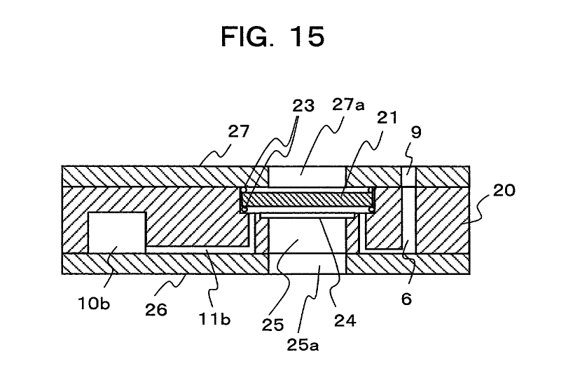

FIGS. 14(a) and 14(b) are explanatory views showing another example of the body used for the test chamber of this embodiment. FIG. 14(a) is a top view, and FIG. 14(b) is a section view taken along the line B-B′ of FIG. 14(a). Moreover, FIG. 15 is a section view showing a structure of the test chamber assembled at the time of measuring. To facilitate understanding of this embodiment, the same function portions of FIGS. 14(a), 14(b) and 15 as those in FIGS. 8 and 9 are denoted by the same reference numerals in FIGS. 8 and 9 and described by use of the same reference numerals.

FIG. 14(a) corresponds to a drawing illustrating FIG. 8(a) more in detail. In the concave portion 22, the O-ring 23, the reagent introduction/suction port 12 and the reagent drain port 13 are disposed. The glass plate 24 exists at the center of a lower portion of the concave portion 22, and the cavity 25 is provided under the concave portion 22.

At the time of measurement, the lower and upper sealing members 26 and 27 are fitted. The opening portions 26a and 27a are respectively provided at the portions corresponding to the positions of the cavity 25 and the glass plate 24. Thus, influences of the lower and upper sealing members 26 and 27 can be removed in the optical measurement. An opaque material can be used for the lower and upper sealing members 26 and 27.

According to the present invention, a stain step can be simply performed without moving a test chamber under a microscope, and treatment of waste liquid is facilitated. Moreover, a specimen after hybridization can be stained simply, and comparison between a gene localization and a cell image can be performed easily.

What is claimed is:

1. A test chamber, comprising:
an observation portion for observing a fixed specimen by use of transmission light;
a plurality of reagent storage portions for storing reagent used for a treatment of the specimen fixed to said observation portion, at least a part of walls of each of said reagent storage portions being made of an elastic material; and
passages for allowing said reagent storage portions and said observation portion to communicate with each other.

2. The test chamber according to claim 1, wherein said observation portion communicates with the open air through a passage having a capacity capable of accepting an excess of the reagent sent from said reagent storage portions to said observation portion and flowing out from said observation portion.

3. The test chamber according to claim 1, wherein said observation portion is sealed by a transparent member, whereby a space filled with the reagent is formed in said observation portion.

4. The test chamber according to claim 1, wherein a transparent plate for fixing the specimen is provided freely detachably from said observation portion.

5. The test chamber according to claim 1, wherein each of said reagent storage portions holds the reagent in a liquid absorption material filled therein.

6. The test chamber according to claim 1, wherein discrimination means for discriminating reagent sets stored in said reagent storage portions are provided.

7. A test chamber, comprising:
an observation portion for observing a fixed specimen by use of transmission light;
a plurality of reagent storage portions for storing reagent used for a treatment of the specimen fixed to said observation portion, at least a part of each of said reagent storage portions being sealed by a stickable material;
a liquid absorber for absorbing the reagent;
passages for allowing said reagent storage portions and said observation portion to communicate with each other; and
a passage for allowing said observation portion and said liquid absorber to communicate with each other.

8. The test chamber according to claim 7, wherein said observation portion is sealed by a transparent member, whereby a space filled with the reagent is formed in said observation portion.

9. The test chamber according to claim 7, wherein a transparent plate for fixing the specimen is provided freely detachably from said observation portion.

10. The test chamber according to claim 7, wherein each of said reagent storage portions holds the reagent in a liquid absorption material filled therein.

11. The test chamber according to claim 7, wherein discrimination means for discriminating reagent sets stored in said reagent storage portions are provided.
